(19) United States
(12) Patent Application Publication
(10) Pub. No.: US 2010/0262254 A1
(43) Pub. Date: Oct. 14, 2010

(54) METATARSAL BONE IMPLANT

(75) Inventors: Bruce R. Lawrence, Oceanside, CA (US); Rebecca Hawkins Wahl, Escondido, CA (US)

Correspondence Address: Roeder & Broder LLP, 5560 Chelsea Avenue, La Jolla, CA, 92037, US

(73) Assignee: Solana Surgical LLC, Oceanside, CA (US)

(21) Appl. No.: 12/421,480

(22) Filed: Apr. 9, 2009

(51) Int. Cl. A61F 2/42

(52) U.S. Cl. 623/21.19

(57) ABSTRACT

A metatarsal implant (212) for insertion into a stem aperture (25) in a metatarsal (14) bone of a foot and to form a joint with the proximal phalanx (16) of the great toe, includes a head (226) and a stem (228). The head (226) includes an articular joint engaging surface (232) and an opposed, bone engaging surface (230). The stem (228) cantilevers away from the bone engaging surface (230), and is adapted to be inserted into the stem aperture (25). The stem (228) can have a stem axis (228A) that is at an angle (234) of between approximately 45 and 75 degrees relative to the bone engaging surface (230). The stem axis (228A) can also be at an angle (266) of between approximately 40 and 80 degrees relative to a tangential reference line (234) drawn from the articular joint engaging surface (232). In one embodiment, the stem axis (228A) is at an angle (266) of between approximately 50 and 70 degrees relative to the tangential reference line (234) of the articular joint engaging surface (232).

### BACKGROUND

It is well known that some people have problems with one or more joints in their feet, with particular problems being seen in the forefoot, or the front of the foot. The forefoot includes the metatarsals and the phalanges, with the joint between these bones commonly referred to as the metatarsal phalangeal joint. The proximal end or base of each of these bones has a smooth articular surface where it forms a joint with the adjacent bone. The base of each of these bones is generally concave in shape. The distal end or head of each of these bones also has a smooth articular surface that is generally convex in shape, so as to enable smooth relative movement between the head of one bone and the base of the adjacent bone. Additionally, near the head of the first metatarsal, on the plantar surface of the foot, are two sesamoid bones, which articulate with the head of the first metatarsal and function as part of the metatarsal phalangeal joint. The sesamoid bones are held in place by tendons and are supported by ligaments, and in combination they are commonly referred to as the sesamoid apparatus. The metatarsal phalangeal joint is capable of motion in two directions, plantar flexion (bending toward the sole of the foot) and dorsiflexion (bending toward the top of the foot), and it also permits abduction (spreading apart) and adduction (bringing together) of the toes.

The majority of disease seen in toe joints affects the head of the first metatarsal. Problems with the metatarsal phalangeal joint of a toe, most commonly with the big toe or great toe, include pain and swelling due to rheumatoid arthritis, hallux limitus (where motion is restricted due to abnormal structure or function), hallux rigidis (where motion is severely restricted or absent), pain due to an impacted joint, joint deterioration or deformation often associated with arthritis, and/or unstable or painful joints due to previous surgeries.

Several procedures have been developed to treat these conditions. For example, implants are often used to obtain pain relief and improve function of the metatarsal phalangeal joint. In the past, reconstruction of the first metatarsal phalangeal joint has been achieved by replacing the phalanx base and/or replacing the metatarsal head. Numerous attempts to replace the head of the metatarsal or its articular surface have been employed with various types of implants.

Unfortunately, problems with previous implants include limited range of motion, implant failure, loosening and displacement of the implant, plantar pain, and disruption of the sesamoid apparatus. Further, implantation of these implants typically requires the removal of significant amounts of healthy bone and/or joint tissue.

### SUMMARY

The present invention is directed toward a metatarsal implant for a foot. The metatarsal implant is designed to be inserted into a stem aperture in a metatarsal bone of the foot and to form a joint with a proximal phalanx of the great toe. The metatarsal implant includes a head and a stem. The head includes an articular joint engaging surface and an opposed, bone engaging surface. The joint engaging surface is adapted to engage the proximal phalanx. The stem cantilevers away from the bone engaging surface, and is adapted to be inserted into the stem aperture.

Uniquely, in certain embodiments, the stem has a stem axis that is at an angle of between approximately 40 and 80 degrees relative to the bone engaging surface. The stem axis can also be at an angle of between approximately 40 and 80 degrees relative to a tangential reference line drawn from the articular joint engaging surface. In one embodiment, the stem axis is at an angle 50 and 70 degrees relative to the tangential reference line of the articular joint engaging surface.

In some embodiments, the articular joint engaging surface has a substantially flat oval mushroom shape. For example, the articular joint engaging surface can include a first depression and a second depression that is positioned substantially opposite of the first depression. In one such embodiment, the first depression and the second depression have a shape that is somewhat similar to a segment of an arc. Further, the articular joint engaging surface can include a first lateral side, a second lateral side, an upper side, and a lower side, wherein the upper side includes the first depression and wherein the lower side includes the second depression.

In another embodiment, the stem can include a proximal section and a distal section. In this embodiment, the proximal section has a cross-section having a first shape and a distal section has a cross-section having a second shape. Moreover, the first shape can be different than the second shape. Further, in one embodiment, the first shape can be substantially non-circular (e.g. rectangular) whereas the second shape can be substantially circular.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a side view of a portion of a foot and a metatarsal implant having features of the present invention;

FIG. 1B is a side view of the portion of the foot illustrated in FIG. 1A;

FIG. 1C is a side view of the portion of the foot and the metatarsal implant of FIG. 1A, with a portion of the foot flexed;

FIG. 1D is a side view of the portion of the foot and the metatarsal implant of FIG. 1A, and FIG. 1D also illustrates an area of variable decompression;

FIG. 2A is a perspective view of an embodiment of a metatarsal implant having features of the present invention;

FIG. 2B is an alternative perspective view of the metatarsal implant illustrated in FIG. 2A;

FIG. 2C is an end view of the metatarsal implant illustrated in FIG. 2A;

FIG. 2D is a side view of the metatarsal implant illustrated in FIG. 2A;

FIG. 2E is an end view of the metatarsal implant illustrated in FIG. 2A;

FIG. 2F is a bottom view of the metatarsal implant illustrated in FIG. 2A;

FIG. 2G is a top view of the metatarsal implant illustrated in FIG. 2A;

FIG. 3A is a perspective view of another embodiment of a metatarsal implant having features of the present invention;

FIG. 3B is an alternative perspective view of the metatarsal implant illustrated in FIG. 3A;

FIG. 3C is an end view of the metatarsal implant illustrated in FIG. 3A;

FIG. 4A is a perspective view of still another embodiment of a metatarsal implant having features of the present invention;

FIG. 4B is an alternative perspective view of the metatarsal implant illustrated in FIG. 4A;

FIG. 4C is an end view of the metatarsal implant illustrated in FIG. 4A.

### DESCRIPTION

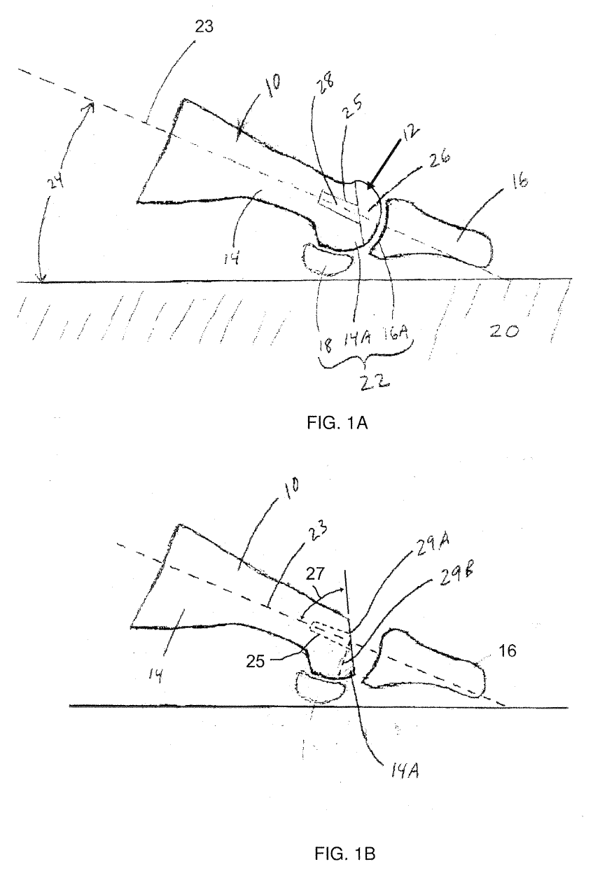

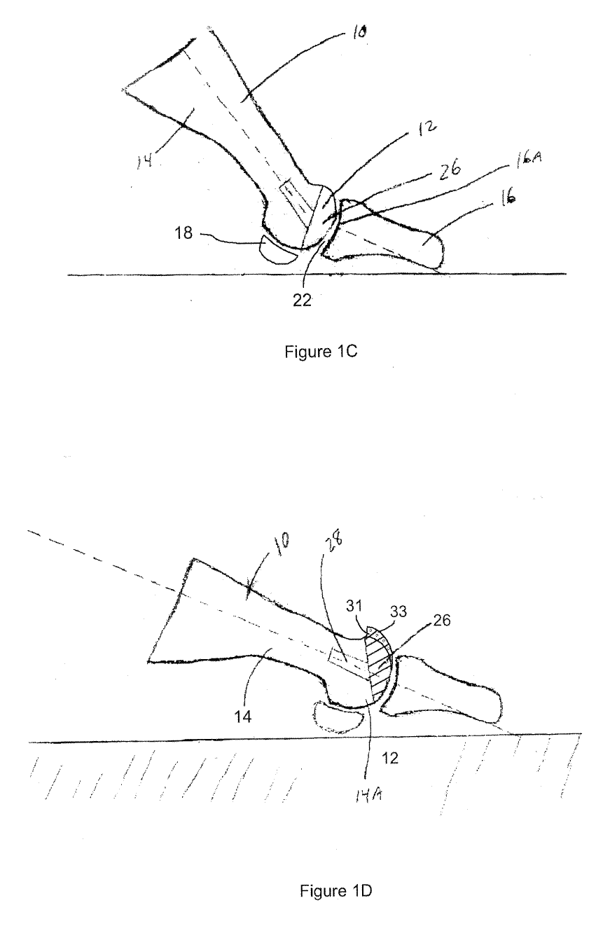

FIG. 1A is a side view of a portion of a foot 10 of a person and a metatarsal implant 12 having features of the present invention. The foot 10 includes a metatarsal 14 having a distal end 14A, a proximal phalanx 16 having a proximal end 16A that is positioned adjacent to the distal end 14A of the metatarsal 14, and a sesamoid apparatus 18 that is positioned substantially below the distal end 14A of the metatarsal 14 and helps to support the metatarsal 14 and the rest of the foot 10 above a surface 20, such as a floor or the ground. The distal end 14A of the metatarsal 14, the proximal end 16A of the proximal phalanx 16, and the sesamoid apparatus 18 cooperate to form a metatarsal phalangeal joint 22.

In FIG. 1A, the foot 10 is positioned substantially flat on the surface 20. In this position, depending upon the shape of the foot 10 of the person, a longitudinal axis 23 (illustrated as a dashed line) of the metatarsal 14 can be inclined at an angle 24 of between approximately 10 and 35 degrees relative to the surface 20. For example, for one person, the longitudinal axis 23 of the metatarsal 14 can be inclined at an angle 24 of approximately twenty-two degrees relative to the surface 20.

As illustrated in FIG. 1A, the metatarsal implant 12 is implanted within a stem aperture 25 that has been drilled along the longitudinal axis 23 within the distal end 14A of the metatarsal 14, adjacent to the proximal end 16A of the proximal phalanx 16 and substantially above the sesamoid apparatus 18. Positioned in this manner, the metatarsal implant 12 effectively forms a part of a metatarsal phalangeal joint 22. The design of the metatarsal implant 12 can be varied depending on the shape of the foot 10 of the person. In this embodiment, the metatarsal implant 12 includes a head 26 and a stem 28 that cantilevers away from the head 26. In one embodiment, the metatarsal implant 12 is sized and shaped to be implanted into the metatarsal of the big toe or great toe, i.e., the first metatarsal, of the foot 10.

As an overview, the metatarsal implant 12 of the present invention allows for a restructuring or replacement of part of the distal end 14A of the metatarsal 14 with minimal bone removal required and without damaging the sesamoid apparatus 18, which, as noted above, helps to support the metatarsal 14 and the rest of the foot 10 above the surface 20. More specifically, the metatarsal implant 12 of the present invention is uniquely designed so that it can be implanted into the distal end 14A of the metatarsal 14 and extend along the longitudinal axis 23 of the metatarsal 14 so as to effectively stabilize the metatarsal 14 and the metatarsal phalangeal joint 22 with minimal bone removal from the metatarsal 14 required and without interfering with the structure, integrity or functioning of the sesamoid apparatus 18. Moreover, the specific design and orientation of the metatarsal implant 12 enables smooth relative motion between the head 26 of the metatarsal implant 12, which is replacing a part of the distal end 14A of the metatarsal 14, and the proximal end 16A of the proximal phalanx 16.

Additionally, the metatarsal implant 12 of the present invention enables the decompressing of the metatarsal phalangeal joint 22, so as to minimize the excessive compression of the metatarsal phalangeal joint 22. Further, because of the unique implant 12 design, the distal end 14A of the metatarsal 14 must be cut at an angle relative to the longitudinal axis 23 of the metatarsal 14 to properly fit the implant. The combination of the bone cut and the implant 12 head shape creates additional space within the joint to lessen compressive forces during dorsiflexion of the joint. The decompression is variable or dynamic in that the greater the dorsiflexion, the greater the decompression. Stated in another fashion, with the design provided herein, there is additional space within the joint 22 that lessens compressive forces during dorsiflexion of the joint.

Further, the metatarsal implant 12 is uniquely shaped and positioned within the metatarsal 14 to minimize, and properly direct, the weight bearing forces that are transmitted through the metatarsal phalangeal joint 22 and onto the metatarsal implant 12. Still further, the metatarsal implant 12 is designed to inhibit rotation of the metatarsal implant 12 when implanted into the metatarsal 14.

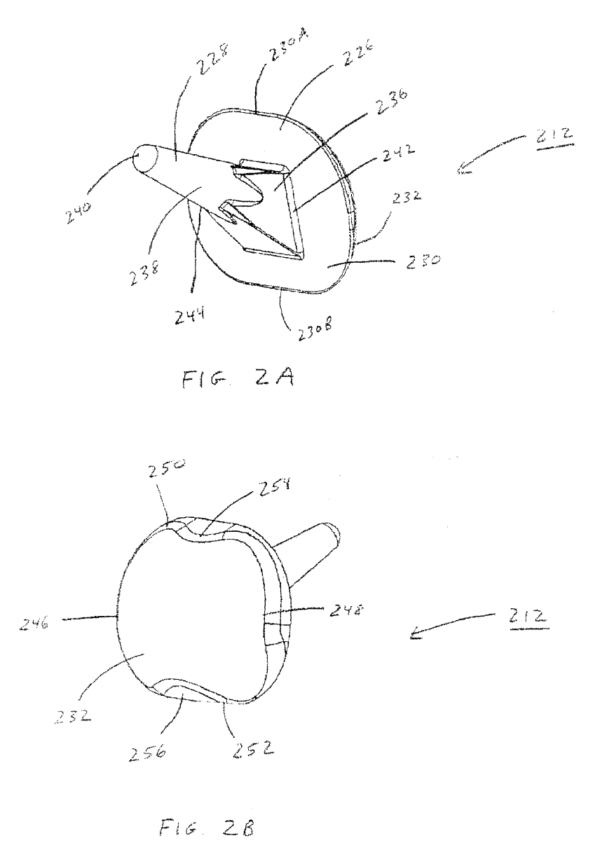

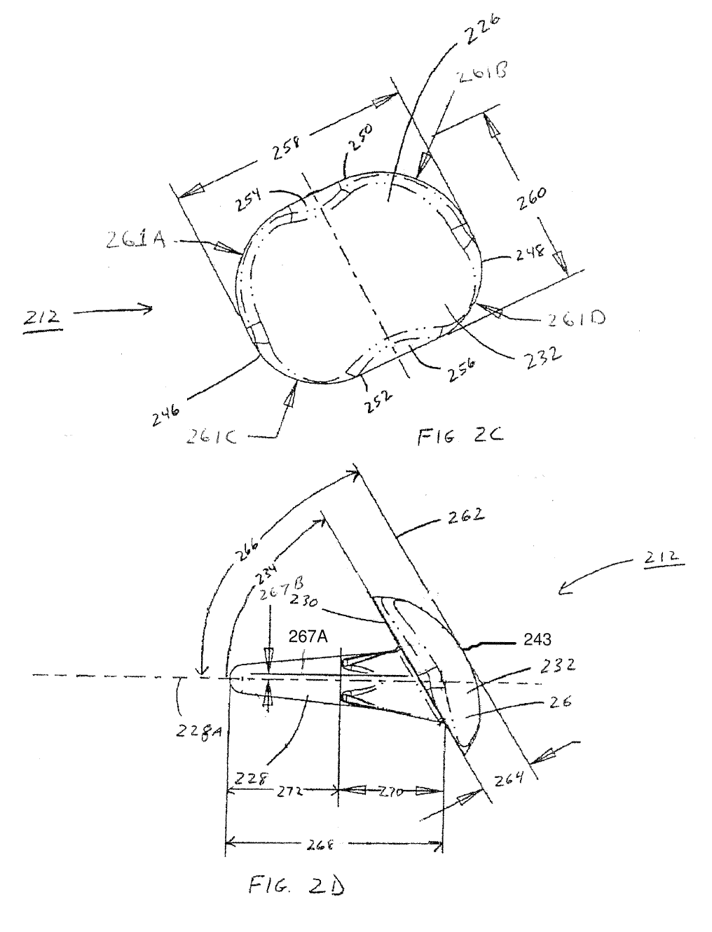

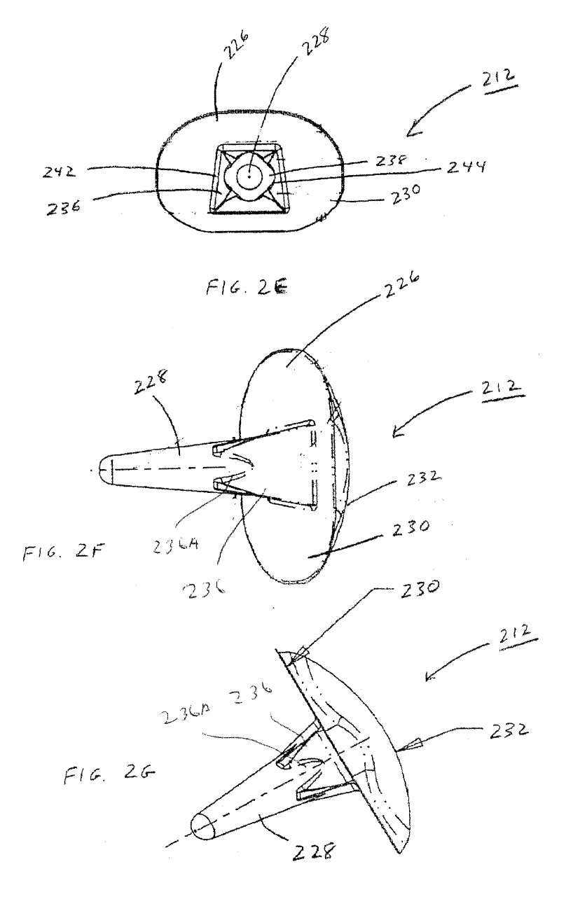

FIG. 1B is a side view of the portion of the foot 10 illustrated in FIG. 1A. More particularly, FIG. 1B illustrates the stem aperture 25 (illustrated in phantom) that has been drilled along the longitudinal axis 23 (illustrated as a dashed line) within the metatarsal 14, and a cut line 29A along which a portion of the distal end 14A of the metatarsal 14 has been cut and removed to allow the necessary spacing for the implanting of the metatarsal implant 12. It should be noted that the cut line 29A is at an angle 27 relative to the longitudinal axis 23 of the metatarsal 14. Because of the angled cut-line 29A, the combination of the bone cut and the implant 12 head shape creates additional space within the joint to lessen compressive forces during dorsiflexion of the joint. It should be noted that the angle 27 of the cut-line 29A should be approximately equal to and match the angle 234 (illustrated in FIG. 2D) of the head 26 relative to the stem 28.

Also illustrated in FIG. 1B is a shaded area 29B (illustrated with “/” cross-hatching) that illustrates the portion of the metatarsal that does not have to be removed because of the unique design of the metatarsal implant 12 (illustrated in FIG. 1A) disclosed herein. Because, the shaded area 29B does not have to be removed, the shaded area 29B is available for interaction with the sesamoid apparatus 18 during movement of the metatarsal relative to the proximal phalanx. Further, as a result thereof, the metatarsal implant 12 is less likely to engage the sesamoid apparatus 18 during movement of the metatarsal relative to the proximal phalanx, and the metatarsal implant 12 is less likely to interfere with the structure, integrity and functioning of the sesamoid apparatus 18. This will result in a more natural functioning of the metatarsal phalangeal joint 22.

FIG. 1C is a side view of the portion of the foot 10 and the metatarsal implant 12 of FIG. 1A, with a portion of the foot 10 flexed, i.e., the metatarsal 14 is inclined relative to the proximal phalanx 16, as occurs during walking. As illustrated, the head 26 of the metatarsal implant 12 is shaped so as to minimize or eliminate any friction or any discomfort between the head 26 and the proximal end 16A of the proximal phalanx 16 within the metatarsal phalangeal joint 22 during flexion of the foot 10. As the foot 10 is flexed, the head 26 of the metatarsal implant 12 rotates relative to the proximal end 16A of the proximal phalanx 16, while still maintaining ample spacing between the head 26 and the proximal end 16A of the proximal phalanx 16, so as to avoid any issues of impingement between the head 26 and the proximal end 16A of the proximal phalanx 16. Additionally, with the unique design of the metatarsal implant 12, flexion of the foot 10 does not result in the head 26 of the metatarsal implant 12 engaging the sesamoid apparatus 18 in any manner. Accordingly, this enables the person with the metatarsal implant 12 to minimize any discomfort within the foot 10 that otherwise may occur during flexion of the foot 10 that occurs during walking and other natural motions of the foot 10.

FIG. 1D is a side view of the portion of the foot 10 and the metatarsal implant 12 of FIG. 1A. FIG. 1D also illustrates a removed area 31 (illustrated with cross-hatching “/”) of bone at the top of the distal end 14A of the metatarsal 14 that was removed so that the metatarsal 14 could receive the implant 12. It should be noted that the removed area 31 illustrates the approximate shape of the distal end 14A of the metatarsal 14 prior to the procedure of preparing the metatarsal 12 for the implant 12.

In this side view of this embodiment, comparing the head 26 of the implant 12 to the shape of the removed area 31, it should be noted that while the shape of the lower (“plantar”) portion of the head 26 corresponds to the shape of the lower (“plantar”) portion of the removed area 31, the upper (“dorsal”) portion of the head 26 is much smaller than the upper (“dorsal”) portion of removed area 31. More specifically, the removed area 31 includes an extra area 33 (illustrated with small circles) that was not mimicked by the shape of the head 26. With this design, as discussed above, the combination of the bone cut and the shape of the head 26 of the implant 12 allows for additional space within the joint to lessen compressive forces during dorsiflexion of the joint.

FIG. 2A is a perspective view of an embodiment of a metatarsal implant 212 having features of the present invention. As shown, the metatarsal implant 212 includes a head 226 and a stem 228 that cantilevers away from the head 226. In certain embodiments, the metatarsal implant 212 is machined from a cast cobalt chrome alloy. Alternatively, the metatarsal implant 212 can be made from a different, relatively lightweight and sturdy material. After machining, the metatarsal implant 212 is polished in certain areas so as to remove any burrs that may exist on the surface of the metatarsal implant 212 which may cause irritations during motion of the foot 10 (illustrated in FIG. 1A) within the metatarsal phalangeal joint 22 (illustrated in FIG. 1A). Finally, the metatarsal implant 212 can be finished with a titanium plasma spray so as to provide rough surfaces throughout the metatarsal stem and bone engaging surface 230 of implant 212.

The head 226 is shaped somewhat similar to a head of a mushroom and includes a bone engaging surface 230 and an opposed articular joint engaging surface 232 (illustrated more clearly in FIG. 2B). The design of the head 226 can be varied depending upon the requirements of the metatarsal implant 212. In one embodiment, the bone engaging surface 230 is generally planar. Further, the perimeter of the bone engaging surface 230 can be shaped to closely match (“approximates”) the shape of the metatarsal 14 at the cut line 29A (illustrated in FIG. 1B). With this design, there is a smooth transition between the implant 12 and the metatarsal 14. This will reduce the likelihood for irritation during movement of the toe and flexing of the joint 22.

Alternatively, the bone engaging surface 230 can have coatings, rough texture and/or contain depression portions or recessed portions so as to allow bone growth into the depression portions or recessed portions in order to further stabilize the positioning of the metatarsal implant 212 within the stem aperture 25 (illustrated in FIG. 1A) that has been drilled into the metatarsal 14 (illustrated in FIG. 1A). However, even in these alternative embodiments, the bone engaging surface 230 still maintains a generally planar surface near the perimeter of the bone engaging surface 230 so as to effectively enable the head 226 to be seated comfortably adjacent to the remaining bone tissue at the distal end 14A (illustrated in FIG. 1A) of the metatarsal 14.

In the embodiment illustrated in FIG. 2A, the bone engaging surface 230 includes an upper edge 230A and a lower edge 230B. The upper edge 230A is positioned toward the top of the distal end 14A of the metatarsal 14 (illustrated in FIG. 1A), and the lower edge 230B is positioned toward the bottom of the distal end 14A of the metatarsal 14 when the metatarsal implant 212 is implanted within the foot 10.

The stem 228 cantilevers away from the bone engaging surface 230 of the head 226 so that a stem axis 228A (illustrated in FIG. 2D) of the stem 228 is positioned at an angle 234 (illustrated in FIG. 2D) relative to the bone engaging surface 230. The stem axis 228A and the stem 228 are angled toward the upper edge 230A of the bone engaging surface 230 and away from the lower edge 230B of the bone engaging surface 230. Uniquely, the stem 228 cantilevers away from the bone engaging surface 230 so that the stem axis 228A of the stem 228 is positioned at an angle 234 other than perpendicular relative to the bone engaging surface 230. This design enables the metatarsal implant 212 to be positioned within the stem aperture 25 (illustrated in FIG. 1A) along the longitudinal axis 23 (illustrated in FIG. 1A) of the metatarsal 14. In one embodiment, the head 226 and the stem 228 are integrally formed together as a single piece during the manufacturing process. Alternatively, the head 226 and the stem 228 can be manufactured separately, with the stem 228 being directly secured to the bone engaging surface 230 of the head 226. In such embodiments, the stem 228 can be secured to the bone engaging surface 230 of the head 226 by gluing, welding, threading or some other method.

In one embodiment, the shape of the stem 228 gradually tapers as the stem 228 moves away from the head 226. The design of the stem 228 can be varied depending upon the requirements of the metatarsal implant 212. As illustrated in FIG. 2A, the stem 228 includes a proximal section 238, a distal section 236 and a tip 240. In one embodiment, the proximal section 238, the distal section 236, and the tip 240 are integrally formed together as a single piece during the manufacturing process. Alternatively, one or more of the proximal section 238, the distal section 236, and the tip 240 can be manufactured separately, and secured together later by gluing, welding or some other method.

In the embodiment illustrated in FIG. 2A, the distal section 236 has a cross-section with a first shape 242 and the proximal section 238 has a cross-section with a second shape 244 that is different than the first shape 242. More particularly, as shown, (i) the distal section 236 is shaped somewhat similar to a truncated rectangular cone and has a cross-section with a substantially non-circular first shape 242 (e.g. rectangular in shape), and (ii) the proximal section 238 is substantially circular cone-shaped having a cross section with a substantially circular second shape 244. Alternatively, the distal section 236 can have a cross-section with a substantially circular first shape 242, and/or the proximal section 238 can have a cross-section with a substantially non-circular second shape 244. Still alternatively, in certain designs, the distal section 236 and the proximal section 238 can have cross-sections wherein the first shape 242 is substantially the same as the second shape 244.

By utilizing a substantially circular second shape 244 of the cross-section of the proximal section 238, as illustrated in the embodiment in FIG. 2A, the metatarsal implant 12 can be easily inserted into the stem aperture 25 (illustrated in FIG. 1A) that has been drilled into the metatarsal 14 (illustrated in FIG. 1A).

By utilizing a substantially non-circular first shape 242 of the cross-section of the proximal section 236, as illustrated in the embodiment in FIG. 2A, the metatarsal implant 212 will be inhibited from rotating within the stem aperture 25 after the metatarsal implant has been inserted into the stem aperture 25 in the metatarsal 14. With the unique design of the joint engaging surface 232, it is important to inhibit rotation of the implant 212 and to insure the proper orientation of the implant 212 for the proper functions of the implant 212.

As shown in FIG. 2A, the tip 240 has a substantially rounded profile. This design is utilized to allow for easy insertion of the metatarsal implant 212 into the stem aperture 25 (illustrated in FIG. 1A).

FIG. 2B is an alternative perspective view of the metatarsal implant 212 illustrated in FIG. 2A. More particularly, FIG. 2B is a perspective view of the metatarsal implant 212 that illustrates many features of the articular joint engaging surface 232. The design of the articular joint engaging surface 232 can be varied depending upon the requirements of the metatarsal implant 212. As illustrated, the articular joint engaging surface 232 has a somewhat flat oval shaped perimeter and includes a first lateral side 246, a second lateral side 248, an upper (“dorsal”) side 250, and a lower (“plantar”) side 252. Additionally, the articular joint engaging surface 232 is also somewhat tapered along the periphery so that the cross-sectional area of the articular joint engaging surface 232 gets smaller as you move away from the opposed bone engaging surface 230 (illustrated in FIG. 2A) along the articular joint engaging surface 232. The upper side 250 is positioned toward the top of the distal end 14A of the metatarsal 14 (illustrated in FIG. 1A), and the lower side 252 is positioned toward the bottom of the distal end 14A of the metatarsal 14 when the metatarsal implant 212 is implanted within the foot 10. Accordingly, the upper side 250 is adjacent to the upper edge 230A (illustrated in FIG. 2A) of the bone engaging surface 230 (illustrated in FIG. 2A), and the lower side 252 is adjacent to with the lower edge 230B (illustrated in FIG. 2A) of the bone engaging surface 230.

Stated in another fashion, the articular joint engaging surface 232 is relatively thick at the center axis of the head, and thins at the dorsal side 250 and the plantar side 252.

The articular joint engaging surface 232 is generally mushroom shaped and can be formed by sweeping a second radius over a first radius. The articular joint engaging surface 232 can have a shape that is similar to a slice of a torus. Further, the perimeter of the articular joint engaging surface 232 is thinned by tapering the perimeter.

In the embodiment illustrated in FIG. 2B, the upper side 250 includes an upper depression 254. Somewhat similarly, the lower side 252 includes a lower depression 256. In this embodiment, because the articular joint engaging surface 232 is oval shaped and is wider than tall, (i) the upper depression 254 is used to thin the articular joint engaging surface 232 near the upper side 250, and (ii) the lower depression 256 is used to thin the articular joint engaging surface 232 near the lower side 252 In one embodiment, the upper depression 254 is shaped as if a small portion of an arc has been removed along the upper side 250 of the articular joint engaging surface 232. Somewhat similarly, the lower depression 256 is shaped as if a small portion of an arch has been removed along the lower side 252 of the articular joint engaging surface 232. The depressions 254, 256 reduce the profile of the articular joint engaging surface 232 near the respective side 250, 252, thereby enabling a more natural functioning of the metatarsal 14 (illustrated in FIG. 1A) during use. Alternatively, the depressions 254, 256 can be designed to have a different shape and/or the articular joint engaging surface 232 can be designed with less than two or more than two depressions 254, 256.

It should be noted that the thinning cuts (e.g. the depressions 254, 256) at the sides 250, 252 can be accomplished with a straight edge rather than a radial cut.

FIG. 2C is an end view of the metatarsal implant 212 illustrated in FIG. 2A. More particularly, FIG. 2C is a view of the metatarsal implant 212 looking straight down onto the head 226 of the metatarsal implant 212 so that only the articular joint engaging surface 232 of the head 226 of the metatarsal implant 212 is visible. As provided above, the articular joint engaging surface 232 is somewhat flat oval shaped and includes the first lateral side 246, the second lateral side 248, the upper side 250, and the lower side 252.

In non-exclusive embodiments, the size of the head 226 of the metatarsal implant 212 can be designed to match the cut surface shape of the metatarsal. The width 258 of the head 226 from the first lateral side 246 to the second lateral side 248 is between approximately 0.6 and 0.9 inches. Alternatively, the head 226 of the metatarsal implant 212 can be designed so that the width 258 of the head 226 from the first lateral side 246 to the second lateral side 248 is less than 0.6 inches or greater than 0.9 inches.

Somewhat similarly, in non-exclusive embodiments, the size of the head 226 of the metatarsal implant 212 can be designed so that a height 260 of the head 226 from the upper side 250 to the lower side 252 is between approximately 0.5 and 0.7 inches. Alternatively, the head 226 of the metatarsal implant 212 can be designed so that the height 260 of the head 226 from the upper side 250 to the lower side 252 is less than 0.5 inches or greater than 0.7 inches.

Further, as illustrated in FIG. 2C, the articular joint engaging surface 232 includes (i) an upper left edge 261A that extends between and connects the upper side 250 and the first lateral side 246; (ii) an upper right edge 261B that extends between and connects the upper side 250 and the second lateral side 248; (iii) a lower left edge 261C that extends between and connects the first lateral side 246 and the lower side 252; and (iv) a lower right edge 261D that extends between and connects the second lateral side 248 and the lower side 252.

In certain embodiments, the upper left edge 261A, the upper right edge 261B, the lower left edge 261C and the lower right edge 261D are substantially rounded in shape as if to form a part of a circle. For example, in non-exclusive embodiments, a radius of the upper left edge 261A can be between approximately 0.2 inches and 0.3 inches; a radius of the upper right edge 261B can be between approximately 0.2 inches and 0.3 inches; a radius of the lower left edge 261C can be between approximately 0.2 inches and 0.3 inches and a radius of the lower right edge 261D can be between approximately 0.2 inches and 0.3 inches. Alternatively, the upper left edge 261A, the upper right edge 261B, the lower left edge 261C and the lower right edge 261D can be designed to have different sizes. Still alternatively, the upper left edge 261A, the upper right edge 261B, the lower left edge 261C and the lower right edge 261D can be designed to have a different shape.

FIG. 2D is a side view of the metatarsal implant 212 illustrated in FIG. 2A. Viewed from the side, as in FIG. 2D, the articular joint engaging surface 232 has a generally rounded profile. Further, the head 26 is thicker in the middle (e.g. head center 243) and thinner near the edges of the head 26 somewhat similar to the head of a mushroom.

Additionally, a tangential reference line 262 can be drawn from the portion of the articular joint engaging surface 232 that is farthest away from the bone engaging surface 230. As illustrated, the tangential reference line 262 is approximately parallel to the generally planar surface of the bone engaging surface 230 of the head 226. Accordingly, a surface height 264 can be measured between the tangential reference line 262 and the bone engaging surface 230. In certain non-exclusive embodiments, the surface height 264 is between approximately 0.1 inches and 0.2 inches. Alternatively, the head 226 of the metatarsal implant 212 can be designed so that the surface height 264 is less than 0.1 inches or greater than 0.2 inches.

Additionally, with the tangential reference line 262 being approximately parallel to the bone engaging surface 230 of the head 226, the angle 234 measured between the stem axis 228A (illustrated as a dashed line) of the stem 228 and the bone engaging surface 230 of the head 226 is substantially the same as an angle 266 measured between the stem axis 228A of the stem 228 and the tangential reference line 262 drawn from the articular joint engaging surface 232 of the head 226.

As provided above, the stem 228 cantilevers away from the bone engaging surface 230 so that the stem axis 228A of the stem 228 is positioned at an angle 234 other than perpendicular relative to the bone engaging surface 230. Somewhat similarly, the stem axis 228A of the stem 228 is also positioned at an angle 266 other than perpendicular relative to the tangential reference line 262. In some embodiments, the angle 234 of the stem axis 228A relative to the bone engaging surface 230 and/or the angle 266 of the stem axis 228A relative to the tangential reference line 262 can be between approximately 40 and 80 degrees depending on the specific anatomy of the metatarsal that the metatarsal implant 212 is being inserted into. In the embodiment illustrated in FIG. 2D, the stem 228 cantilevers away from the head 226 such that the angle 234 of the stem axis 228A relative to the upper side 250 of the bone engaging surface 230 is between approximately 50 and 70 degrees. Somewhat similarly, the stem 228 cantilevers away from the head 226 such that the angle 266 of the stem axis 228A relative to the tangential reference line 262 is approximately 50 and 70 degrees. Thus, the stem 228 is effectively positioned relative to the head 226 so that the stem axis 228A is at an angle of approximately 50 and 70 degrees relative to the articular joint engaging surface 232 of the head 226.

In alternative, non-exclusive embodiments, the stem axis 228A can be approximately 40, 45, 50, 55, 60, 65, 70, 75, or 80 degrees.

The stem 228 cantilevers away from the bone engaging surface 230 of the head 226 in an area that is substantially centrally located on the bone engaging surface 230 of the head 226. However, as illustrated in FIG. 2D, a center axis line 267A drawn parallel to the stem axis 228A from the midpoint of the bone engaging surface 230 between the upper edge 230A (illustrated in FIG. 2A) and the lower edge 230B (illustrated in FIG. 2A) shows that the stem 228 is slightly offset an offset distance 267B along the bone engaging surface 230 from the upper edge 230A to the lower edge 230B. Stated another way, the stem 228 cantilevers away from the bone engaging surface 230 so that the stem 228 at the point where it engages the bone engaging surface 230 is slightly closer to the bottom edge 230B than to the upper edge 230A.

In non-exclusive embodiments, the metatarsal implant 212 can be designed so that the offset distance 267B is between approximately 0.01 inches and 0.03 inches. Alternatively, the metatarsal implant 212 can be designed so that the offset distance 267B is less than 0.01 inches or greater than 0.03 inches.

The offset allows the implant head 226 to be correctly positioned on the cut bone surface of the metatarsal 14 so that the inferior edge of the implant 12 does not overhang one of the sesamoid grooves and restrict joint motion. The offset also allows for anatomic variations of individual metatarsal heads. The grooves on the inferior aspect of the metatarsal head for the articulation of the sesamoid bones may not be symmetrical.

Further, as provided above, the stem 228 includes the distal section 236, the proximal section 238 and the tip 240. In non-exclusive embodiments, a stem length 268 of the stem 228 can be between approximately 0.6 and 0.8 inches. Alternatively, the stem length 268 can be less than 0.6 inches or greater than 0.8 inches. Since the tip 240 constitutes such a small portion of the overall stem length 268, the remainder of this discussion will be pursued as if the distal section 238 was of a length equal to the combined length of the distal section 238 and the tip 240.

As shown in the embodiment illustrated in FIG. 2D, a distal length 270 of the distal section 236 can make up approximately 48 percent of the stem length 268, with a proximal length 272 of the proximal section 238 making up the other 52 percent of the stem length 268. In alternative embodiments, the distal length 270 can vary from approximately 20 percent of the stem length 268 to approximately 80 percent of the stem length 268. More particularly, the distal length 270 can make up approximately 20, 30, 40, 50, 60, 70 or 80 percent of the stem length 268, or some other value. Similarly, the proximal length 272 can vary from approximately 20 percent of the stem length 268 to approximately 80 percent of the stem length 268. More particularly, the proximal length 272 can make up approximately 20, 30, 40, 50, 60, 70 or 80 percent of the stem length 268, or some other value. For example, in one embodiment, the distal length 270 makes up approximately 20 percent of the stem length 268 while the proximal length 272 makes up approximately 80 percent of the stem length 268. Still alternatively, the distal length 270 can make up approximately 40 percent of the stem length 268 while the proximal length 272 makes up approximately 60 percent of the stem length 268. Additionally, the distal length 270 can make up approximately 60 percent of the stem length 268 while the proximal length 272 makes up approximately 40 percent of the stem length 268. Further, the distal length 270 can make up approximately 80 percent of the stem length 268 while the proximal length 272 makes up approximately 20 percent of the stem length 268.

FIG. 2E is an end view of the metatarsal implant 212 illustrated in FIG. 2A. More particularly, FIG. 2E is a view of the metatarsal implant 212 looking directly down the stem 228 toward the head 226 of the metatarsal implant 212. In this embodiment, the stem 228 cantilevers away from the bone engaging surface 230 of the head 226 in an area that is substantially centrally located on the bone engaging surface 230 of the head 226, although it is slightly offset as noted above with regard to the description provided of FIG. 2D.

As noted above in relation to FIG. 2A, and as illustrated more clearly in FIG. 2E, the distal section 236 is designed with a cross-section having the first shape 242, and the proximal section 238 is designed with a cross-section having the second shape 244. In alternative embodiments, the second shape 244 can be designed to be different than the first shape 242, or the second shape 244 can be designed to have substantially the same shape as the first shape 242.

In certain embodiments, the first shape 242 of the distal section 236 has a non-circular cross-section. The non-circular cross-section is designed to keep the metatarsal implant 212 from rotating once it has been inserted or implanted into the metatarsal 14 (illustrated in FIG. 1A). For example, in the embodiment illustrated in FIG. 2B, the first shape 242 of the distal section 236 has a substantially square shaped cross-section. Alternatively, the stem 228 can be designed so that the first shape 242 of the distal section 236 has a different shaped cross-section. In some non-exclusive examples, the first shape 242 of the distal section 236 can be designed to have a substantially rectangular shaped cross-section, a substantially square shaped cross-section, a substantially diamond shaped cross-section or a substantially hexagonal shaped cross-section.

Additionally, the second shape 244 of the proximal section 238, as illustrated, can have a substantially circular cross-section. Alternatively, the stem 228 can be designed so that the second shape 244 of the proximal section 238 has a different shaped cross-section. In some non-exclusive examples, the second shape 244 of the proximal section 238 can be designed to have a substantially rectangular shaped cross-section, a substantially square shaped cross-section, a substantially diamond shaped cross-section, or a substantially hexagonal shaped cross-section.

FIG. 2F is a bottom view of the metatarsal implant 212 illustrated in FIG. 2A. Additionally, FIG. 2G is a top view of the metatarsal implant 212 illustrated in FIG. 2A. In particular, FIGS. 2F and 2G illustrate views wherein the stem 228 appears to cantilever perpendicularly away from the head 226. Accordingly, in the embodiment illustrated in the Figures, the unique angle of the stem 228 relative to the bone engaging surface 230 and/or the articular joint engaging surface 232 of the head 226 is only fully visible from a side view perspective.

Additionally, as illustrated in FIGS. 2F and 2G, each side of the distal section 236 can include a somewhat “V” shaped slot 236A that allows for bone growth into the proximal section 236. This will further inhibit rotation of the implant 212.

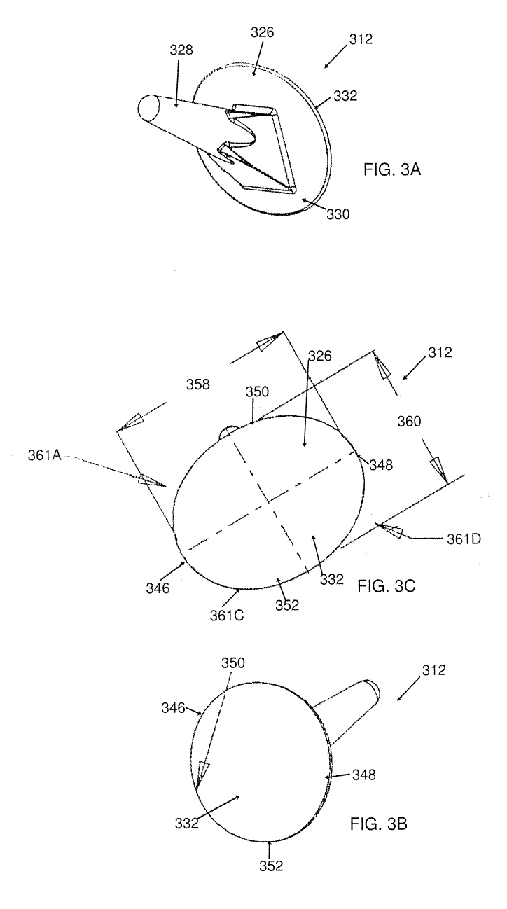

FIG. 3A is a perspective view of another embodiment of a metatarsal implant 312 having features of the present invention. Similar to the metatarsal implant 212 as described in FIGS. 2A-2G, the metatarsal implant 312 illustrated in FIG. 3A includes a head 326 and a stem 328 that cantilevers away from the head 326. The stem 328 is substantially similar in design, shape, manufacture, positioning and functioning to the stem 228 disclosed in the embodiment as described in FIG. 2A-2G. Accordingly, a detailed description of the stem 328 will not be repeated.

Additionally, the head 326 includes a bone engaging surface 330 and an opposed articular joint engaging surface 332 (illustrated more clearly in FIG. 3B). The bone engaging surface 330 of the head 326 is substantially similar in design and functioning as the bone engaging surface 230 disclosed in the embodiment as described in FIGS. 2A-2G. Accordingly, a detailed description of the bone engaging surface 330 will not be repeated.

FIG. 3B is an alternative perspective view of the metatarsal implant 312 illustrated in FIG. 3A. More particularly, FIG. 3B is a perspective view of the metatarsal implant 312 that illustrates many features of the articular joint engaging surface 332. The design of the articular joint engaging surface 332 can be varied depending upon the requirements of the metatarsal implant 312. As illustrated, the articular joint engaging surface 332 has a design and orientation that is somewhat similar to the articular joint engaging surface 232 as described in relation to the embodiment illustrated in FIGS. 2A-2G. For example, the articular joint engaging surface 332 has a somewhat flat oval shaped perimeter and includes a first lateral side 346, a second lateral side 348, an upper (“dorsal”) side 350, and a lower (“plantar”) side 352. Additionally, the articular joint engaging surface 332 is also somewhat tapered along the periphery so that the cross-sectional area of the articular joint engaging surface 332 gets smaller as you move away from the opposed bone engaging surface 330 (illustrated in FIG. 3A) along the articular joint engaging surface 332. Stated in another fashion, the articular joint engaging surface 332 is relatively thick at the center axis of the head 326, and thins at the dorsal side 350 and the plantar side 352. Further, the perimeter of the articular joint engaging surface 332 is thinned by tapering the perimeter.

The upper side 350 is positioned toward the top of the distal end 14A of the metatarsal 14 (illustrated in FIG. 1A), and the lower side 352 is positioned toward the bottom of the distal end 14A of the metatarsal 14 when the metatarsal implant 312 is implanted within the foot 10.

As noted above, the metatarsal implant 312 illustrated in FIG. 3B includes the articular joint engaging surface 332 that is somewhat similar in shape to the articular joint engaging surface 232 as described in relation to the embodiment illustrated in FIGS. 2A-2G. In contrast, however, in the embodiment illustrated in FIG. 3B, the articular joint engaging surface 332 is designed without the upper depression 254 (illustrated in FIG. 2B) and the lower depression 256 (illustrated in FIG. 2B).

FIG. 3C is an end view of the metatarsal implant 312 illustrated in FIG. 3A. More particularly, FIG. 3C is a view of the metatarsal implant 312 looking straight down onto the head 326 of the metatarsal implant 312 so that only the articular joint engaging surface 332 of the head 226 of the metatarsal implant 212 is visible. As provided above, the articular joint engaging surface 332 is somewhat flat oval shaped and includes the first lateral side 346, the second lateral side 348, the upper side 350, and the lower side 352.

The size of the head 326 can be varied to suit the requirements of the metatarsal implant 312. In certain non-exclusive embodiments, a width 358 of the head 326 from the first lateral side 346 to the second lateral side 348 can be between approximately 0.6 and 0.9 inches. Alternatively, the head 326 of the metatarsal implant 312 can be designed so that the width 358 of the head 326 from the first lateral side 346 to the second lateral side 348 is less than 0.6 inches or greater than 0.9 inches.

Somewhat similarly, in non-exclusive embodiments, the size of the head 326 of the metatarsal implant 312 can be designed so that a height 360 of the head 326 from the upper side 350 to the lower side 352 is between approximately 0.5 and 0.7 inches. Alternatively, the head 326 of the metatarsal implant 312 can be designed so that the height 360 of the head 326 from the upper side 350 to the lower side 352 is less than 0.5 inches or greater than 0.7 inches.

Further, as illustrated in FIG. 3C, the articular joint engaging surface 332 includes (i) an upper left edge 361A that extends between and connects the upper side 350 and the first lateral side 346; (ii) an upper right edge 361B that extends between and connects the upper side 350 and the second lateral side 348; (iii) a lower left edge 361C that extends between and connects the first lateral side 346 and the lower side 352; and (iv) a lower right edge 361D that extends between and connects the second lateral side 348 and the lower side 352.

In certain embodiments, the upper left edge 361A, the upper right edge 361B, the lower left edge 361C and the lower right edge 361D are substantially rounded in shape as if to form a part of a circle. For example, in non-exclusive embodiments, a radius of the upper left edge 361A can be between approximately 0.2 inches and 0.3 inches; a radius of the upper right edge 361B can be between approximately 0.2 inches and 0.3 inches; a radius of the lower left edge 361C can be between approximately 0.2 inches and 0.3 inches and a radius of the lower right edge 361D can be between approximately 0.2 inches and 0.3 inches. Alternatively, the upper left edge 361A, the upper right edge 361B, the lower left edge 361C and the lower right edge 361D can be designed to have different sizes. Still alternatively, the upper left edge 361A, the upper right edge 361B, the lower left edge 361C and the lower right edge 361D can be designed to have a different shape.

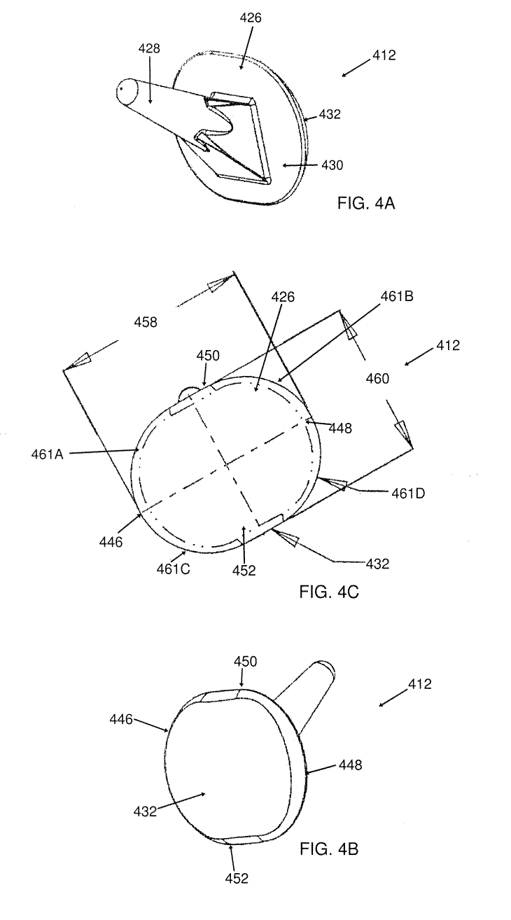

FIG. 4A is a perspective view of still another embodiment of a metatarsal implant 412 having features of the present invention. Similar to the metatarsal implant 212 as described in FIGS. 2A-2G, the metatarsal implant 412 illustrated in FIG. 4A includes a head 426 and a stem 428 that cantilevers away from the head 426. The stem 428 is substantially similar in design, shape, manufacture, positioning and functioning to the stem 228 disclosed in the embodiment as described in FIG. 2A-2G. Accordingly, a detailed description of the stem 428 will not be repeated.

Additionally, the head 426 includes a bone engaging surface 430 and an opposed articular joint engaging surface 432 (illustrated more clearly in FIG. 4B). The bone engaging surface 430 of the head 426 is substantially similar in design and functioning as the bone engaging surface 230 disclosed in the embodiment as described in FIGS. 2A-2G. Accordingly, a detailed description of the bone engaging surface 430 will not be repeated.

FIG. 4B is an alternative perspective view of the metatarsal implant 412 illustrated in FIG. 4A. More particularly, FIG. 4B is a perspective view of the metatarsal implant 412 that illustrates many features of the articular joint engaging surface 432. The design of the articular joint engaging surface 432 has a design that is somewhat different than the articular joint engaging surface 232 as described in relation to FIGS. 2A-2G. As illustrated, the articular joint engaging surface 432 has a somewhat flat elliptical perimeter and includes a first lateral side 446, a second lateral side 448, an upper (“dorsal”) side 450, and a lower (“plantar”) side 452. Additionally, the articular joint engaging surface 432 is also somewhat tapered along the periphery so that the cross-sectional area of the articular joint engaging surface 432 gets smaller as you move away from the opposed bone engaging surface 430 (illustrated in FIG. 4A) along the articular joint engaging surface 432. Stated in another fashion, the articular joint engaging surface 432 is relatively thick at the center axis of the head 426, and thins at the dorsal side 450 and the plantar side 452. Further, the perimeter of the articular joint engaging surface 432 is thinned by tapering the perimeter.

The upper side 450 is positioned toward the top of the distal end 14A of the metatarsal 14 (illustrated in FIG. 1A), and the lower side 452 is positioned toward the bottom of the distal end 14A of the metatarsal 14 when the metatarsal implant 412 is implanted within the foot 10.

FIG. 4C is an end view of the metatarsal implant 412 illustrated in FIG. 4A. More particularly, FIG. 4C is a view of the metatarsal implant 412 looking straight down onto the head 426 of the metatarsal implant 412 so that only the articular joint engaging surface 432 of the head 426 of the metatarsal implant 412 is visible. As provided above, the articular joint engaging surface 432 is somewhat flat elliptical shaped and includes the first lateral side 446, the second lateral side 448, the upper side 450, and the lower side 452.

The size of the head 426 can be varied to suit the requirements of the metatarsal implant 412. In certain non-exclusive embodiments, a width 458 of the head 426 from the first lateral side 446 to the second lateral side 448 can be between approximately 0.6 and 0.9 inches. Alternatively, the head 426 of the metatarsal implant 412 can be designed so that the width 458 of the head 426 from the first lateral side 446 to the second lateral side 448 is less than 0.6 inches or greater than 0.9 inches.

Somewhat similarly, in non-exclusive embodiments, the size of the head 426 of the metatarsal implant 412 can be designed so that a height 460 of the head 426 from the upper side 450 to the lower side 452 is between approximately 0.5 and 0.7 inches. Alternatively, the head 426 of the metatarsal implant 412 can be designed so that the height 460 of the head 426 from the upper side 450 to the lower side 452 is less than 0.5 inches or greater than 0.7 inches.

Further, as illustrated in FIG. 4C, the articular joint engaging surface 432 includes (i) an upper left edge 461A that extends between and connects the upper side 450 and the first lateral side 446; (ii) an upper right edge 461B that extends between and connects the upper side 450 and the second lateral side 448; (iii) a lower left edge 461C that extends between and connects the first lateral side 446 and the lower side 452; and (iv) a lower right edge 461D that extends between and connects the second lateral side 448 and the lower side 452.

In certain embodiments, the upper left edge 461A, the upper right edge 461B, the lower left edge 461C and the lower right edge 461D are substantially rounded in shape as if to form a part of a circle. For example, in non-exclusive embodiments, a radius of the upper left edge 461A can be between approximately 0.2 inches and 0.3 inches; a radius of the upper right edge 461B can be between approximately 0.2 inches and 0.3 inches; a radius of the lower left edge 461C can be between approximately 0.2 inches and 0.3 inches and a radius of the lower right edge 461D can be between approximately 0.2 inches and 0.3 inches. Alternatively, the upper left edge 461A, the upper right edge 461B, the lower left edge 461C and the lower right edge 461D can be designed to have different sizes. Still alternatively, the upper left edge 461A, the upper right edge 461B, the lower left edge 461C and the lower right edge 461D can be designed to have a different shape.

While the particular metatarsal implant 12 as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A metatarsal implant for insertion into a stem aperture in a metatarsal bone of a foot, the metatarsal implant forming a joint with a proximal phalanx of the toe, the metatarsal implant comprising:
a head including an articular joint engaging surface and an opposed, bone engaging surface, the joint engaging surface being adapted to engage the proximal phalanx; and
a stem that cantilevers away from the bone engaging surface, the stem being adapted to be inserted into the stem aperture, the stem having a stem axis that is at an angle of between approximately 40 and 80 degrees relative to the bone engaging surface.

2. The metatarsal implant of claim 1 wherein the stem axis is at an angle of between approximately 40 and 80 degrees relative to a tangential reference line of the articular joint engaging surface.

3. The metatarsal implant of claim 2 wherein the stem axis is at an angle of between approximately 50 and 70 degrees relative to the tangential reference line of the articular joint engaging surface.

4. The metatarsal implant of claim 1 wherein the bone engaging surface has a substantially flat oval footprint to match a cut surface of the metatarsal and the head is thicker near a head center of the head and thinner near a dorsal edge and near a plantar edge of the head.

5. The metatarsal implant of claim 4 wherein the articular joint engaging surface is formed by sweeping a second radius over a first radius.

6. The metatarsal implant of claim 4 wherein the articular joint engaging surface includes a first lateral side, a second lateral side, an upper side, and a lower side, wherein the upper side includes the first depression and wherein the lower side includes the second depression.

7. The metatarsal implant of claim 6 wherein the second depression is larger than the first depression.

8. The metatarsal implant of claim 1 wherein the stem includes a proximal section with a cross-section having a first shape and a distal section with a cross-section having a second shape, wherein the first shape is different than the second shape.

9. The metatarsal implant of claim 8 wherein the first shape is substantially non-circular, and wherein the second shape is substantially circular.

10. A metatarsal implant for insertion into a stem aperture in a metatarsal bone of a foot, the metatarsal implant forming a joint with a proximal phalanx of the toe, the metatarsal implant comprising:
a head including an articular joint engaging surface and an opposed bone engaging surface, the joint engaging surface being adapted to engage the proximal phalanx, wherein the articular joint engaging surface has a substantially flat oval shape; and
a stem that cantilevers away from the bone engaging surface, the stem being adapted to be inserted into the stem aperture.

11. The metatarsal implant of claim 10 wherein the bone engaging surface has a substantially flat oval footprint to match a cut surface of the metatarsal and the head is thicker near the center of the head and thinner near a dorsal edge and near a plantar edge of the head.

12. The metatarsal implant of claim 10 wherein the articular joint engaging surface is formed by sweeping a second radius over a first radius.

13. The metatarsal implant of claim 11 wherein the articular joint engaging surface includes a first lateral side, a second lateral side, an upper side, and a lower side, wherein the upper side includes the first depression and wherein the lower side includes the second depression.

14. The metatarsal implant of claim 10 wherein the stem has a stem axis that is at an angle of between approximately 40 and 80 degrees relative to a tangential reference line of the articular joint engaging surface.

15. The metatarsal implant of claim 14 wherein the stem axis is at an angle of approximately 60 degrees relative to the tangential reference line of the articular joint engaging surface.

16. The metatarsal implant of claim 10 wherein the stem includes a proximal section with a cross-section having a first shape and a distal section with a cross-section having a second shape, wherein the first shape is different than the second shape.

17. The metatarsal implant of claim 16 wherein the first shape is substantially non-circular, and wherein the second shape is substantially circular.

18. A metatarsal implant for insertion into a stem aperture in a metatarsal bone of a foot, the metatarsal implant forming a joint with a proximal phalanx of the toe, the metatarsal implant comprising:
a head including an articular joint engaging surface and an opposed bone engaging surface, the joint engaging surface being adapted to engage the proximal phalanx; and
a stem that cantilevers away from the bone engaging surface, the stem being adapted to be inserted into the stem aperture, the stem including a proximal section with a cross-section having a first shape and a distal section with a cross-section having a second shape, wherein the first shape is different than the second shape.

19. The metatarsal implant of claim 18 wherein the first shape is substantially non-circular, and wherein the second shape is substantially circular.

20. The metatarsal implant of claim 18 wherein the stem has a stem axis that is at an angle of between approximately 40 and 80 degrees relative to a tangential reference line of the articular joint engaging surface.

21. The metatarsal implant of claim 20 wherein the stem axis is at an angle of between approximately 50 and 70 degrees relative to the tangential reference line of the articular joint engaging surface.

22. The metatarsal implant of claim 18 wherein the articular joint engaging surface has a substantially flat oval shape with a first depression and a second depression that is positioned substantially opposite of the first depression.
